(19) United States
(12) Patent Application Publication
(10) Pub. No.: US 2011/0054028 A1
(43) Pub. Date: Mar. 3, 2011

(54) SUPPLEMENT FOR REDUCING HIV TRANSMISSION AND METHOD THEREOF

(75) Inventor: Kate Hsu, Taipei City (TW)

(73) Assignee: MACKAY MEMORIAL HOSPITAL, Taipei City (TW)

(21) Appl. No.: 12/729,217

(22) Filed: Mar. 22, 2010

(30) Foreign Application Priority Data
Aug. 27, 2009 (TW) 098128831

(51) Int. Cl. A61K 31/202; A61P 31/18

(52) U.S. Cl. 514/560

(57) ABSTRACT

There is an urgent need for a method to reduce the risk of mother-to-child transmission (MTCT) of HIV during breastfeeding in HIV pandemic regions. The present invention provides a supplement for reducing the chance of transmitting HIV during MTCT and method thereof. The supplement comprising arachidonic acid (AA) alone, or a mixture of AA and DHA at safe intake doses. The supplement is provided to either the breastfeeding mothers or the babies to maintain cell membrane potential polarization through activation of the background potassium channels, thereby reducing the risk of HIV transmission.

### BACKGROUND OF THE INVENTION

1. Field of the Invention

The present invention is related to a supplement, in particular to a supplement for reducing the chance of transmitting human immunodeficiency virus (HIV) infection and method thereof.

2. The Prior Arts

Breastfeeding by human immunodeficiency virus (HIV)-positive mothers is an unavoidable practice in some very poor countries. For example, breastfeeding accounts for up to 40% of mother-to-child transmission (MTCT) of HIV in sub-Saharan Africa. Although many organizations such as WHO strongly advises against breastfeeding by HIV-positive mothers, replacement feeding in the pandemic, low-income countries is not improved primarily because of the lack of clean water.

Recent nutritional studies show that some natural components in breast milk, such as arachidonic acid (AA), linolenic acid and so on, are beneficial in reducing the risk of MTCT of HIV. It has been suggested that long-chain polyunsaturated fatty acids (LC-PUFAs) act as natural, protective ingredients against HIV transmission through inhibiting HIV activities or enhancing the CD4⁺ cell responses.

The abovementioned long-chain polyunsaturated fatty acids contain at least 2 double bonds, and the number on omega-3 (ω3), omega-6 (ω6) and Omega-9 (ω9) following “omega-” represents the position of the first double bond from the terminal methyl group of a molecule. Omega-3 (ω3) and omega-6 (ω6) fatty acids are unsaturated “Essential Fatty Acids” (EFAs) that cannot be derived from other fatty acid by human metabolism and must be absorbed from one's diet.

Arachidonic acid is an essential omega-6 fatty acid in human. Arachidonic acid is widespread in neutral fat, milk fat, swine fat, bovine fat, and serum phospholipids. In addition, arachidonic acid (AA) is the most abundant PUFA in human, especially in the brain (40-50% of the brain LC-PUFA) and nervous systems (up to 70% in the periphery). The content of plasma arachidonic acid in an individual is up to 400 mg/L. Metabolites of AA in the brain have important effects on the central nervous system, particularly on adjustment of transmembrane signals, release of neurotransmitters, development of brain, enhancement of intelligence, increase of the number and the length of dendrites, and formation of myelination.

Omega-3 fatty acids, including Alpha-linolenic acid, eicosapentaenoic acid (EPA) and docosahexaenoic acid (DHA), are important for human nutrition. DHA is a major component of the cell membrane, and is important for visual and brain functions. DHA is anti-inflammatory and lowers the blood lipid. DHA also has beneficial effects on development of fetus and prevention of cardiovascular and Alzheimer's diseases.

In summary, LC-PUFAs are well known for their cellular protective activities against tissue lesions, e.g. during the events of ischemia, strokes or seizures. The mechanism through which LC-PUFAs stimulate the cellular protection machinery primarily involves modulation or activation of two-pore-domain potassium (two-pore K⁺, K_{2P}, or KCNK) channels. The extensive KCNK channel family comprises 18 gene members, and is ubiquitously expressed in a variety of cell types, including the major HIV target, CD4⁺ T cells.

The main function of KCNK channels is to stabilize the cell membrane potential by drawing it toward the equilibrium potential for K⁺ (i.e. near −90 mV in most physiologic conditions). Thus, the expression level of endogenous K_{2P} channels is a key determinant of cell viability and excitability. It has been demonstrated that during traumatic episodes of ischemia, activation of KCNK channels by LC-PUFAs could drive membrane potential hyperpolarization and suppress harmful excitability or secretion.

Notably, secretion of HIV viral particles from infected cells could be similarly suppressed by the hyperpolarizing activity of KCNK channels. The host KCNK channel is capable of restricting the release of HIV viral particles from infected cells. The KCNK channel limits viral spread by counteracting the activity of an HIV-encoded protein named Vpu, whose main function is to enhance the efficiency of viral particle release by up to 100-fold. Because Vpu bears a channel-like structure, this small viral protein has the tendency to self-oligomerize or to promiscuously oligomerize with homologous host channel subunits like KCNK to destroy the channel function of KCNK.

From previous studies, the protein-protein interaction between host KCNK3 and Vpu was identified in heterologous expression systems (i.e. cervical cancer cells HeLa and human embryonic kidney cells HEK-293), as well as in the HIV-infected human CD4⁺ cells. The functional consequences of their protein-protein interaction are mutually destructive: Vpu accelerates degradation of the host KCNK3 channel to abolish the background K⁺ conductance in HIV-infected cells, while endogenous KCNK3 expression restrains Vpu-mediated virus release.

Therefore, the rate of HIV particle release in infected host cells should be decelerated if KCNK channels can be regulated or activated. It would be desirable to achieve a composition and method to stabilize the cell membrane potential through regulation or activation of KCNK channels to reduce the risk of HIV transmission.

### SUMMARY OF THE INVENTION

In order to reduce the release of viral particles from HIV-infected cells, a composition to maintain the cell membrane potential should be provided. A primary objective of the present invention therefore is to provide a supplement to reduce the risk of HIV transmission.

Another objective of the present invention is to provide a method for maintaining the polarized membrane potential of a cell.

The other objective of the present invention is to provide a method for reducing the risk of mother-to-child transmission (MTCT) of HIV.

To achieve the object described above, the present invention is to provide a supplement for reducing HIV transmission during the breastfeeding period. The supplement comprising arachidonic acid (AA) alone, or a mixture of AA and docosahexaenoic acid (DHA) in a ratio (AA-DHA mixture), and the effective concentration of AA alone is 0.006-0.15% (w/w), and the effective concentration of the AA-DHA mixture is 0.001-0.005% (w/w). For example, the ratio of AA and DHA in the AA-DHA mixture can be 2:1 (w/w). Arachidonic acid (AA) alone, or together with docosahexaenoic acid (DHA) at the safe intake doses for human, stabilize the membrane potential via activation of KCNK channels such as KCNK3 or KCNK9 in CD4⁺ T cells to reduce the risk of HIV transmission. The supplement can be provided to either the breastfeeding mothers or the babies during breastfeeding.

To achieve the objectives described above, the present invention is to provide a method for maintaining polarization of cell membrane potential of a cell by providing a dietary supplement that activates the background potassium channels in CD4⁺ T cells, such as KCNK3, stabilizes membrane potential polarization and lowers the rates of HIV particle release and HIV transmission.

To achieve the objective described above, the present invention is to provide a method that reduces the risk of mother-to-child transmission (MTCT) of HIV, comprising administering to a subject in need thereof an effective amount of the above-mentioned supplement during breastfeeding period to inhibit HIV release, wherein the subject is a breastfeeding mother or a baby. The effective amount of AA alone is 0.3 to 7 mg/kg, and the effective amount of the AA-DHA mixture is 0.12-0.2 mg/kg.

According to the present invention, the release or transmission of HIV virus can be inhibited efficiently by stabilizing membrane potential polarization through maintaining or up-regulating the expression of endogenous background K⁺ channels (i.e. KCNK channels). Therefore, arachidonic acid (AA) and docosahexaenoic acid (DHA) can be incorporated into a dietary supplement, and through supplementation, to maintain the activity of KCNK channels and membrane polarization in order to inhibit the release of HIV particles. In one embodiment, the supplementation may be beneficial in suppression of HIV spread during breastfeeding in the AIDS pandemic regions.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

FIG. 1 shows the relationship between release of HIV-1 particles and cell membrane potential.

FIG. 2 shows the effects of DHA and AA to the knockdown of KCNK3/9 channels.

FIG. 3 shows the suppression of HIV-1 release by AA. The untreated HeLa cell is set at 100% as control (dashed line).

FIG. 4 shows a flowchart listing the steps of using the supplementation in the present invention.

### DETAILED DESCRIPTION OF THE PREFERRED EMBODIMENT

### Definitions

The term “long-chain polyunsaturated fatty acids (LC-PUFA)”, as used herein, is defined as unsaturated fatty acids with at least 2 double bonds in the long chain, with omega-3 and omega-6 fatty acid families being important to human bodies.

The term “essential fatty acid (EFA)”, as used herein, is defined as fatty acids that cannot be synthesized in human, and therefore must be obtained from natural foods, mostly vegetables and seafood.

The term “mother-to-child transmission (MTCT)”, as used herein, is defined as transmission from an infected mother to a child.

The term “small interfering RNA (siRNA)”, as used herein, is defined as small pieces of double-stranded (ds) RNA, usually about 20-25 nucleotides long and involved in specific gene regulating functions.

The term “knock down”, as used herein, is defined as reducing or inhibiting the expression of a target gene by siRNA.

The term “wild-type”, as used herein, is defined as the individuals showing the highest frequency among the colonies.

The term “mutant”, as used herein, is defined as an individual having a new genetic character arising from artificial mutation.

The term “excitability”, as used herein, is defined as changes in membrane potential in response to stimuli.

### Example 1

### HIV-1 Particle Release and Membrane Potential Depolarization

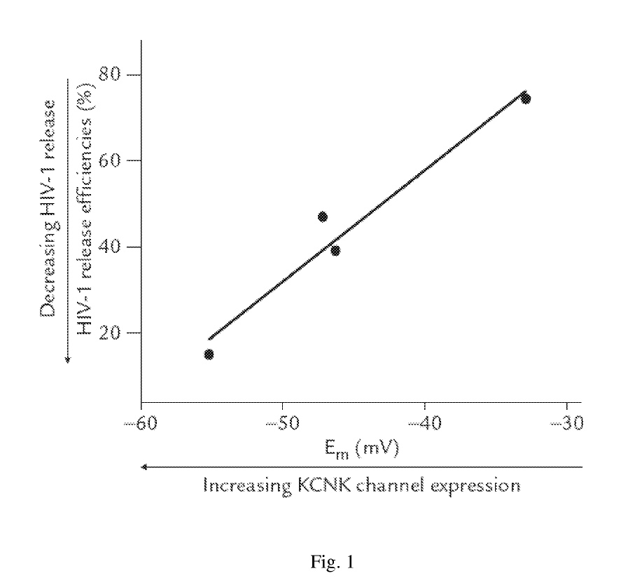

FIG. 1 shows the relationship between HIV-1 particle release and membrane potential depolarization. Each dot represents an averaged value for the dual effects of KCNK3 or one of its functionally distinct mutants on membrane potentials (x-axis) and on HIV-1 particle release efficiency (y-axis). Cell membrane potentials (Em) in a Ringer-like solution were determined by whole-cell patch-clamp recordings. HIV-1 particle release was determined by quantifying the concentration of HIV structural protein p24 using ELISA, which gives the number of secreted viral particles. The inhibitory effects of KCNK3 and its channel mutants on viral release were compared with the single expression of HIV-1 (set at 100%) in HeLa cells.

The cell membrane potentials could be maintained at about −40 mV to −50 mV when HIV-1 and KCNK3 channels were co-expressed heterologously in cervical cancer HeLa cells, and the HIV-1 particle release was suppressed to about 50% with KCNK3 expression (the middle points in FIG. 1).

Cell membrane potentials became hyperpolarized and the rate of HIV-1 particle release was further suppressed to below 20%, when a hyperactive KCNK3 mutant in HeLa cells was expressed (the lower left point in FIG. 1).

On the other hand, a nonfunctional KCNK3 channel mutant exerted neither any hyperpolarizing effects on the membrane potential nor any inhibitory effects on HIV-1 particle release when heterologously expressed (the upper right point in FIG. 1).

An inverse relationship between the efficiency of HIV-1 particle release and the stability of the cell membrane potential maintained by the background potassium channels was shown based on the abovementioned results. The linear fit between the x and the y parameters indicates a direct relationship between membrane potential depolarization and the efficiency of HIV-1 particle release (as indicated by the horizontal and vertical arrows in FIG. 1).

Therefore, the efficiency of HIV-1 particle release was directly proportional to the degree of membrane potential depolarization. The viral release efficiency was from high to low when the membrane potential was from depolarization to hyperpolarization. In other words, the release of HIV viral particles can be slowed down if the expression of KCNK channel is not affected by the virus and is capable of maintaining membrane potential polarization.

### Example 2

### The Effects of a Mixture of Docosahexaenoic Acid (DHA) and Arachidonic Acid (AA) on Membrane Potential Polarization

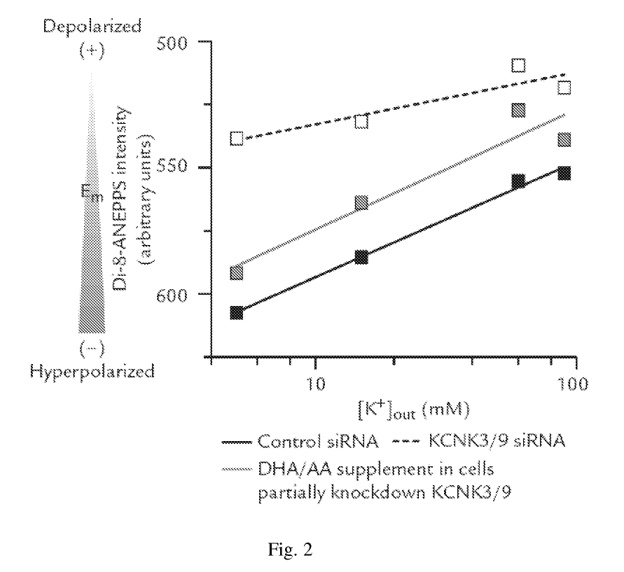

Referring to FIG. 2, the inhibitory effect of arachidonic acid (AA) and docosahexaenoic acid (DHA) on the channel activity of KCNK3 and KCNK9 (KCNK3/9) in HeLa cells was shown.

To mimic the depolarized state of HIV-infected cells, endogenous KCNK3 and KCNK9 channels (KCNK3/9) in HIV-1-susceptible cells (i.e. human CD4⁺ T cells; HeLa cells) were partially knocked down using a low dose of the specific small interfering RNA (siRNA). DHA and AA, each at a concentration equivalent to the recommended dietary intake (1-3.6 μM in cell culture), were supplemented in the form of an arachidonic acid (AA)-docosahexaenoic acid (DHA) mixture with a 2:1 ratio (w/w). The AA-DHA mixture was found to restore the activities of endogenous KCNK channels that were partially knocked down. The activity of endogenous KCNK channels was assessed in terms of cell membrane potential at various concentrations of extracellular K⁺ ([K⁺]ₒᵤₜ). Cells were labeled with polarization-sensitive fluorescent dye di-8-ANEPPS (Invitrogen), which responds to changes in the local electric field strength across the cell membrane. The fluorescent signals were detected by flow cytometry.

In the absence of KCNK knockdown (“control siRNA”), cell membrane potentials were directly correlated with extracellular K⁺ content.

In the group treated with KCNK3/9 siRNA, endogenous KCNK3/9 was partially knocked down to mimic the depolarized state of HIV infected cells. The correlation of cell membrane potential and extracellular K⁺ content was relatively diminished in this group (with a flattened slope). Partial knockdown of endogenous KCNK3/9 reduced the dependency of cell membrane potential on extracellular K⁺ concentrations; the reduced sensitivity to extracellular K⁺ was similar to that in the HIV-infected cells. Most importantly, the knockdown of endogenous KCNK3/9 substantially depolarized cell membrane potential, reducing the electrical constraint on virus release.

On the other hand, membrane potential hyperpolarization in these KCNK3/9-knockdown cells could be restored by supplementation with AA and DHA, to a large extent. The supplementation also activated KCNK channels in the infected cells, and restored the membrane potential sensitivity to [K⁺]ₒᵤₜ, as demonstrated by a slope comparable to that of the control samples, thereby maintaining membrane potential polarization and halting the rate of HIV-1 release.

Thus, AA and DHA were capable of stimulating endogenous KCNK channels in the cells most susceptible to HIV-1 infection and restoring membrane potential polarization, thereby inhibiting the release of HIV-1 particles.

It's known that the membrane potential polarization and other vital homeostatic functions are disrupted for the purpose of viral spread when CD4⁺ T cells are infected with HIV. An infected cell gradually loses its polarizing membrane potential until cell death. However, if the expression of background K⁺ or KCNK channels is maintained or up-regulated during HIV infection, viral secretion and spread could conceivably be suppressed by the hyperpolarizing membrane potentials as described in Example 1.

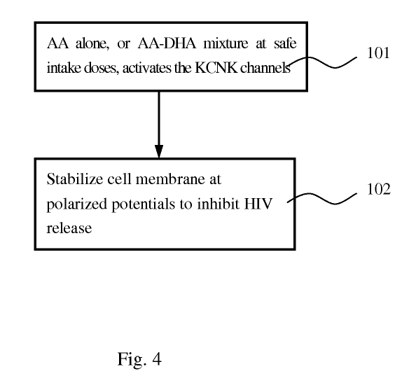

According to Example 2, AA and DHA could activate endogenous KCNK channels in the infected cells, restore the membrane sensitivity to extracellular K⁺ ([K⁺]ₒᵤₜ), maintain membrane potential polarization, and further decelerate the release of HIV-1. Therefore, supplementation of the AA-DHA mixture should be able to restore KCNK channel activities and membrane potential polarization. The supplement containing the AA-DHA mixture was developed, and can be supplemented as described in FIG. 4.

### Example 3

### The Effects of Arachidonic Acid (AA) Alone on HIV-1 Viral Release

HeLa cells at 50% confluency in P25 flasks were transiently transfected with 2-3 μg of one of the HIV-1 proviral constructs (pNL4-3; pNL4-3/Udel). One-tenth of the culture media was retrieved from each sample and filtered through a 0.22-μm syringe filter at each time point. Cells were collected at the end and subjected to whole-cell lysis by 1% SDS. The viral content in culture media and in cell lysates was determined by Coulter HIV-1 p24 Antigen Assay kit. Virus release was compared to that from the control (co-transfection of pNL4-3 and pCGI). Data are expressed as mean±SEM from 8 independent experiments.

For the inhibitory effects of K_{2P} channel, different quantities (0.02-10 μM) of arachidonic acid (Sigma) were tested in HeLa cell culture at the 24^{th} hour post-transfection of NL4-3, and one-tenth of the media was retrieved and filtered at each time point. Viral release was compared to that of the untreated HeLa cell set at 100% (control).

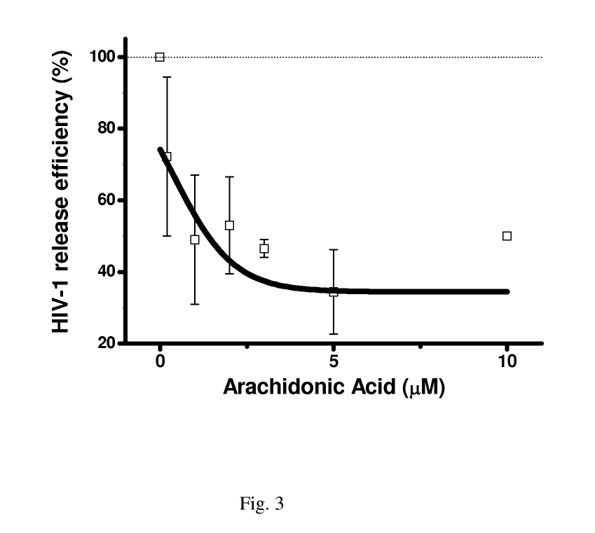

Referring to FIG. 3, the inhibitory effects of HIV-1 viral release in HeLa cells at the 4^{th} hour after arachidonic acid (AA) supplementation were shown. Arachidonic acid (AA) at submicromolar concentrations could suppress viral release in HeLa cells expressing NL4-3. Moreover, 50-60% of the release inhibition could be achieved by AA at 0.5×10⁻⁷˜10⁻⁶ M.

The supplementation for reducing MTCT of HIV comprises AA alone or AA-DHA mixture at safe intake doses for human, as shown in FIG. 4. The effective concentration of AA alone is about 0.006-0.15% (w/w), and the effective concentration of the AA-DHA mixture is about 0.001-0.005% (w/w). Besides, the effective amount of AA alone administrating to breastfeeding mothers or babies is about 0.3 to 7 mg/kg and the effective amount of AA-DHA mixture is 0.12-0.2 mg/kg. These concentrations of the supplement are effective in reducing HIV transmission and safe to human without affecting one's immune system in the long run.

The ratio of AA and DHA can be 2:1 (w/w) in the AA-DHA mixture. The supplement is provided to either the breastfeeding mothers or the babies to activate the background potassium or KCNK channels (Step 101). The supplement can be added directly to the breast milk when provided to babies.

Polarization of the cell membrane potential can be maintained to inhibit the release of HIV particles and reduce the risk of HIV transmission by activating the endogenous KCNK channels in babies (Step 102).

What is claimed is:

1. A supplement for reducing the risk of HIV transmission, comprising arachidonic acid (AA) alone or a mixture of AA and docosahexaenoic acid (DHA) in a ratio (AA-DHA mixture), wherein the effective concentration of AA alone is 0.006-0.15% (w/w), and the effective concentration of the AA-DHA mixture is 0.001-0.005% (w/w).

2. The supplement as claimed in claim 1, wherein the supplement is ingested during the breastfeeding period.

3. The supplement as claimed in claim 1, wherein arachidonic acid (AA) alone, or the AA-DHA mixture activates background potassium channels in a cell and stabilizes membrane potential polarization.

4. The supplement as claimed in claim 3, wherein the background potassium channels primarily comprise KCNK3 and KCNK9 channels.

5. The supplement as claimed in claim 3, wherein the cell is a human cell susceptible to HIV infection.

6. The supplement as claimed in claim 5, wherein the human cell is a CD4⁺ T cell.

7. The supplement as claimed in claim 1, wherein the supplement is added into the breast milk for babies.

8. The supplement as claimed in claim 1, wherein the preferred ratio of arachidonic acid (AA) and docosahexaenoic acid (DHA) in the AA-DHA mixture is 2:1 (w/w).

9. A method for polarizing cell membrane potential, comprising providing the supplement as claimed in claim 1 to activate endogenous background potassium channels of a cell and stabilize membrane potential polarization in order to inhibit HIV release.

10. The method as claimed in claim 9, wherein the background potassium channels primarily comprise KCNK3 channel and KCNK9 channel.

11. The method as claimed in claim 9, wherein the cell is a human cell susceptible to HIV infection.

12. The method as claimed in claim 11, wherein the human cell is a CD4⁺ T cell

13. The method as claimed in claim 9, wherein the supplement is added into the breast milk for babies.

14. The method as claimed in claim 9, wherein the method is applied during the breastfeeding period.

15. The method as claimed in claim 9, wherein the preferred ratio of arachidonic acid (AA) and docosahexaenoic acid (DHA) in the AA-DHA mixture is 2:1 (w/w).

16. A method for reducing the risk of mother-to-child transmission (MTCT) of HIV, comprising administering to a subject in need thereof an effective amount of the supplement as claimed in claim 1 during the breastfeeding period to inhibit HIV release, wherein the subject is a breastfeeding mother or a baby.

17. The method as claimed in claim 16, wherein the effective amount of AA alone is 0.3 to 7 mg/kg and the effective amount of the AA-DHA mixture is 0.12-0.2 mg/kg.

18. The method as claimed in claim 16, wherein the preferred ratio of arachidonic acid (AA) and docosahexaenoic acid (DHA) in the AA-DHA mixture is 2:1 (w/w).
